(19) United States
(12) Patent Application Publication
(10) Pub. No.: US 2016/0113772 A1
(43) Pub. Date: Apr. 28, 2016

(54) SPINAL IMPLANT SYSTEM AND METHOD

(71) Applicant: Warsaw Orthopedic, Inc., Warsaw, IN (US)

(72) Inventors: Jeremy J. Rawlinson, Memphis, TN (US); Gary S. Lindemann, Collierville, TN (US)

(21) Appl. No.: 14/523,522

(22) Filed: Oct. 24, 2014

(51) Int. Cl. A61F 2/44; A61F 2/46

(52) U.S. Cl. CPC A61F 2/44; A61F 2/4611; A61F 2002/30932

(57) ABSTRACT

A method comprising the steps of: providing at least one bone growth modification implant; and disposing the entire bone growth modification implant within a selected portion of a vertebra of a spine. Systems and devices are disclosed.

### TECHNICAL FIELD

The present disclosure generally relates to medical devices for the treatment of musculoskeletal disorders, and more particularly to a surgical system including a spinal implant and a method for deformity correction.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, plates and fasteners, for stabilization of a treated section of a spine, This disclosure describes an improvement over these prior art technologies.

### SUMMARY

In one embodiment, a method for treating a spine is provided. The method comprising the steps of: providing at least one bone growth modification implant; and disposing the entire bone growth modification implant within a selected portion of a vertebra of a spine. In some embodiments, systems and devices are provided.

### BRIEF DESCRIPTION OF THE DRAWINGS

The present disclosure will become more readily apparent from the specific description accompanied by the following drawings, in which:

FIG. 1 is a break away perspective view of components of one embodiment of a system in accordance with the principles of the present disclosure;

FIG. 2 is a plan view of the components shown in FIG. 1 disposed with vertebrae;

FIG. 3 is a plan view of components of one embodiment of a system in accordance with the principles of the present disclosure disposed with vertebrae;

FIG. 4 is a break away perspective view of the vertebrae shown in FIG.

FIG. 5A is a plan view of components of one embodiment of a system in accordance with the principles of the present disclosure disposed with vertebrae;

FIG. 5B is a plan view of components of one embodiment of a system in accordance with the principles of the present disclosure disposed with vertebrae

FIG. 6 is an axial view of components of one embodiment of a system in accordance with the principles of the present disclosure disposed with vertebrae;

FIG. 7 is a side view of the components and vertebrae shown in FIG. 6;

FIG. 8 is a side cross section view of components of one embodiment of a system in accordance with the principles of the present disclosure;

FIG, 9 is an axial view of components of one embodiment of a system in accordance with the principles of the present disclosure disposed with vertebrae; and

FIG. 10 is a plan view of components of one embodiment of a system in accordance with the principles of the present disclosure disposed with vertebrae.

### DETAILED DESCRIPTION

The exemplary embodiments of the spinal implant system and method are discussed in terms of medical devices for the treatment of musculoskeletal disorders and more particularly, in terms of a surgical system and method for treatment of a spine disorder. In some embodiments, the spinal implant system and method may be employed in applications such as correction of deformities, such as, for example, scoliosis.

For example, the present spinal implant system and method can include injecting material delivered into a first side, such as, for example, a convex side of a spine that is curved due to scoliosis. In some embodiments, while the material may be injected into a first side of each of a plurality of vertebrae to prevent growth of vertebrae of the first side, the system allows for growth and adjustments to a second side, such as, for example, a concave side of the plurality of vertebrae.

In one embodiment, the spinal implant system includes a device for injecting material directly into a vertebral body at a location of growth plates, such as, for example, a patient suffering from early onset scoliosis. In some embodiments, the device injects material to hinder and/or slow and/or stop growth on the convex side of the spine.

In one embodiment, the spinal implant system is configured to arrest growth on the convex side of the spine while allowing the concave side of the spine to grow. In one embodiment, the spinal implant system is configured to correct a lateral deformity. In some embodiments, the spinal implant system is configured to correct multiple direction deformities.

In one embodiment, the spinal implant system includes a method of minimally invasive altering of the epiphyseal cartilage area above the growth plate. In one embodiment, the method includes utilizing an injection device and an injection material or solution delivered from a posterior or lateral approach to inhibit and/or accelerate growth of a vertebral body. In one embodiment, the spinal implant system is configured for insertion of the injection material or solution in different quadrants of the vertebral body to create asymmetric growth and correct three dimensional deformities. In one embodiment, the spinal implant system includes an injection material or solution having a low viscosity, such as, for example, a hydrogel that is configured to absorb water and swell thereby creating a pressure above the growth plate. In one embodiment, the low viscosity material is bone cement, such as, for example, poly (methyl methacrylate) (PMMA) that is configured to set and stiffen an area above the growth plate.

In one embodiment, the spinal implant system includes a device, such as, for example, a porous hypodermic needle having porosity in a single plane to deliver a low viscosity injection material or solution in a flat plane. In one embodiment, the spinal implant system includes a device, such as, for example, a balloon angioplasty device. In one embodiment, the spinal implant system includes an expandable metallic mesh, such as, for example, a stent. In one embodiment, the spinal implant system includes an atherectomy device, such as, for example, a specialized catheter. In one embodiment, the spinal implant system comprises a stent including sharp teeth disposed in a single plane.

In one embodiment, the spinal implant system is employed with a method of treating scoliosis in a growing spine using a cryogenic probe. In some embodiments, the method employs a navigated thora-scopic approach. In some embodiments, the cryogenic probe ablates growth plates on a convex side of a spine. In some embodiments, this configuration allows the concave side of the spine to grow and straighten the spine. In some embodiments, the system and method are employed such that alternating convex growth plates are treated to provide selective growth on the convex side of the spine to allow a child continued growth while selectively inhibiting growth for correction. In some embodiments, the method includes facilitating subsequent and separate treatment and/or correction of the spine.

In some embodiments, one or all of the components of the spinal implant system may be disposable, peel-pack, pre-packed sterile devices. One or all of the components of the spinal implant system may be reusable. The spinal implant system may be configured as a kit with multiple sized and configured components.

In some embodiments, the present disclosure may be employed to treat spinal disorders, such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system and method may be employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be employed with procedures for treating the lumbar, cervical, thoracic and pelvic regions of a spinal column. The present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure, It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment, It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

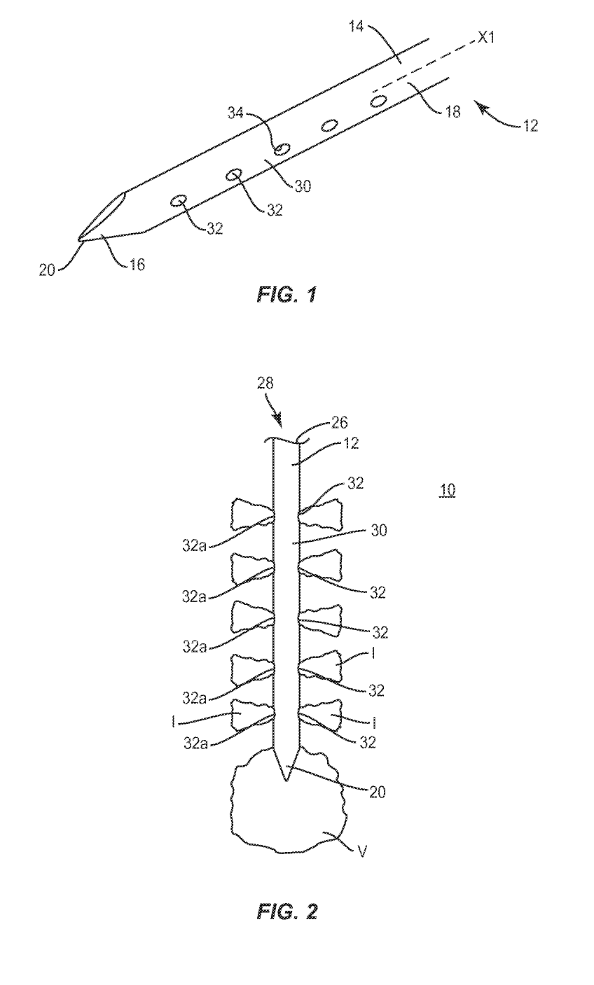

The following discussion includes a description of a spinal implant system, related components and methods for employing the spinal implant system. Alternate embodiments are also disclosed. Reference is made in detail to the exemplary embodiments of the present disclosure, which are illustrated in the accompanying figures. Turning to FIGS. 1 and 2, there is illustrated components of a spinal implant system, such as, for example, a spinal correction system 10 in accordance with the principles of the present disclosure.

The components of system 10 can be fabricated from biologically acceptable materials suitable for medical applications, including metals, synthetic polymers, ceramics and bone material and/or their composites. For example, the components of system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, super elastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Octane Orthobiologics, Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is employed, for example, with an open, mini-open or minimally invasive surgical technique to inject a bone growth modification implant I into vertebrae. System 10 includes a surgical instrument, such as, for example, an injection device. An injection device, such as, for example, an intra-vertebra needle 12 extends between an end 14 and an end 16. Needle 12 defines a longitudinal axis X1. In some embodiments, needle 12 can be variously configured, such as, for example, tubular, oval, oblong, triangular, square, polygonal, irregular, uniform, non-uniform, variable, hollow and/or tapered. End 14 includes an opening 18 configured for connection with a dispenser of implant I (not shown). End 16 includes a pointed tip 20 configured to penetrate and/or engage tissue.

Needle 12 includes a surface 26 that defines a cavity, such as, for example, a passageway 28 configured for disposal of implant I. Needle 12 includes a surface 30 that defines at least one lateral opening 32. In some embodiments, lateral openings 32 may be disposed at alternate orientations, relative to axis X1, such as, for example, transverse, perpendicular and/or other angular orientations such as acute or obtuse, co-axial and/or may be offset or staggered. In some embodiments, surface 26 and/or surface 30 may include alternate surface configurations, such as, for example, rough, arcuate, undulating, mesh, porous, semi-porous, dimpled and/or textured.

In one embodiment, as shown in FIG. 1, needle 12 includes a plurality of openings 32 linearly disposed between end 14 and end 16. In one embodiment, as shown in FIG. 2, needle 12 includes a first plurality of openings 32 disposed in a linear orientation along a first side of needle 12 and a second plurality of openings 32a disposed in a linear orientation along a second side of needle 12. In some embodiments, openings 32, 32a may be disposed at alternate orientations, relative to axis X1, such as, for example, transverse, perpendicular and/or other angular orientations such as acute or obtuse, co-axial and/or may be offset or staggered.

In some embodiments, surface 26 and/or surface 30 may include alternate surface configurations, such as, for example, rough, arcuate, undulating, mesh, porous, semi-porous, dimpled and/or textured. Each opening 32 includes a surface 34 that extends between surface 26 and surface 30. Openings 32 are configured to deliver implant I such that implant I flows through openings 32 under pressure. In some embodiments, needle 12 is connected to a pump to force implant I comprising fluid and/or other fluids through openings 32. In some embodiments, needle 12 includes a syringe configuration with a piston to deliver implant I through openings 32 under pressure. In some embodiments, this pressure can be controlled during delivery such that the pressure is not static. In some embodiments, implant I flows through openings 32 into a convex side of each of a plurality of vertebrae to prevent growth of the convex side of the vertebrae of a selected section of the spine. In some embodiments, system 10 allows for growth and adjustments to a second side, such as, for example, a concave side of the plurality of vertebrae for a correction treatment to treat various spine pathologies, such as, for example, adolescent idiopathic scoliosis and Scheuermann's kyphosis.

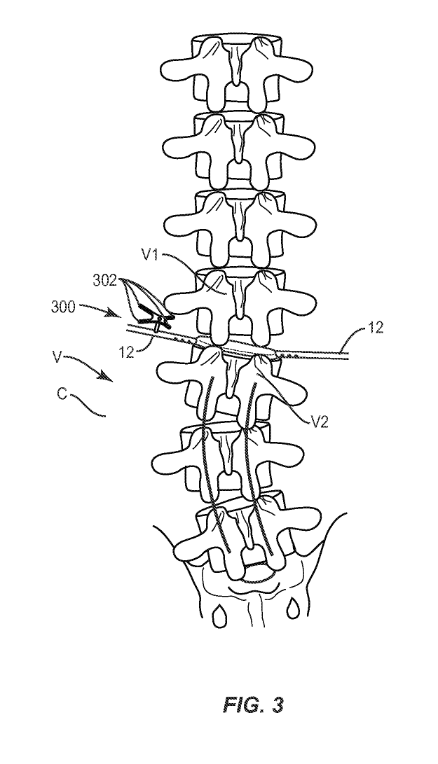

In one embodiment, as shown in FIG. 3, needle 12 includes image guidance and/or surgical navigation to monitor, maintain, adjust and/or confirm disposal, delivery and/or alignment of the components of system 10 adjacent to a surgical site. For example, the surgical navigation components of system 10 facilitate placement of needle 12. The surgical navigation components of system 10 include an emitter 300 configured to generate a signal representative of a position of needle 12 connected therewith, for example, adjacent to a surgical site. In some embodiments, emitter 300 may include one or a plurality of emitters. In one embodiment, emitter 300 is shaped substantially like the Greek letter pi and comprises four spaced apart emitters 302, for generating a signal representing the trajectory of needle 12 relative to a portion of a patient's anatomy and the depth of needle 12 adjacent to a surgical site. In one embodiment, emitter 300 includes at least one light emitting diode. In some embodiments, emitter 300 may include other tracking devices capable of being tracked by a corresponding sensor array, such as, for example, a tracking device that actively generates acoustic signals, magnetic signals, electromagnetic signals, radiologic signals. In some embodiments, emitter 300 may be removably attached to needle 12. In some embodiments, emitter 300 may be integrally formed with needle 12 such that needle 12 is a monolithic, unitary body.

In some embodiments, system 10 includes a tracking device (not shown) having an emitter array including one or a plurality of emitters that generate signals representing the position of various body reference points of the patient's anatomy. A sensor (not shown) receives signals from emitter 300 and the array. The sensor communicates with a processor (not shown), such as, for example, a digitizer control unit, which processes the signals from emitter 300 and the array to provide information regarding the trajectory of needle 12 relative to a portion of the patient's anatomy and the depth of needle 12 adjacent to a surgical site. The processor sends this information to a monitor, which provides a visual representation of the position of needle 12 adjacent to a surgical site to allow the medical practitioner to guide needle 12 to a desired location within the patient's anatomy.

The monitor is configured to generate an image from a data set stored in a controller, such as, for example, a computer. In some embodiments, the data set may be generated preoperatively using scanning techniques, such as, for example, a CAT scanner or MRI scanner. The image data set includes reference points for at least one body part, such as, for example, the spine of a patient, which has a fixed spatial relation to the body part. The processor is connected to the monitor, under control of the computer, and to needle 12.

The sensor receives and triangulates signals generated by emitter 300 and the array to identify the relative position of each of the reference points and needle 12. The processor and the computer modify the image data set according to the identified relative position of each of the reference points during the procedure. The position and trajectory of needle 12 provided by emitter 300 and the array is processed by the processor and the computer and is visually displayed against the preoperative image data set stored in the computer to provide the medical practitioner with a visual representation of the trajectory of needle 12 relative to a portion of the patient's anatomy and the depth of needle 12 within the patient's anatomy. See, for example, similar surgical navigation components and their use as described in U.S. Pat. Nos. 6,021,343, 6,725,080, 6,796,988, the entire contents of each of these references being incorporated by reference herein.

In one embodiment, implant I includes a growth inhibiting implant. In some embodiments, implant I comprises an absorbent fluid that is configured to apply pressure to a portion of the vertebra above a growth plate of the vertebra, such as, for example, altering the epiphyseal cartilage area above the growth plate to inhibit growth of a vertebral body. In some embodiments, implant I includes an injection material that creates a pressure above the growth plate to inhibit growth. In one embodiment, implant I includes bone cement configured to stiffen the vertebra and/or to stiffen tissue above the growth plate to inhibit growth. In some embodiments, implant I includes a bone growth promoting implant, such as, for example, bone graft and/or one or a plurality of therapeutic agents and/or pharmacological agents for release, including sustained release, to treat, for example, pain, inflammation and degeneration.

In assembly, operation and use, correction system 10, similar to the systems and methods described herein, is employed with a surgical procedure, such as, for a correction treatment to treat adolescent idiopathic scoliosis and/or Scheuermann's kyphosis of a spine. In some embodiments, one or all of the components of spinal correction system 10 can be delivered or implanted as a pre-assembled device or can be assembled in situ. Spinal correction system 10 may be completely or partially revised, removed or replaced.

For example, as shown in FIG. 3, system 10 can be employed with a surgical correction treatment of an applicable condition or injury of an affected section of a spinal column and adjacent areas within a body along a convex side C of vertebrae V. In some embodiments, spinal correction system 10 may be employed with one or a plurality of vertebrae.

In use, to treat a selected section of vertebrae V, a medical practitioner obtains access to a surgical site including vertebrae V along a posterior percutaneous pathway P (for example, as shown in FIG. 9 for vertebra V2). In one embodiment, access to a surgical site including vertebrae V is along a lateral percutaneous pathway. A medical practitioner obtains access to a surgical site including vertebrae V through an incision and retraction of tissues. In some embodiments, spinal correction system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of system 10. A preparation instrument (not shown) can be employed to prepare tissue surfaces of vertebrae V, as well as for aspiration and irrigation of a surgical region.

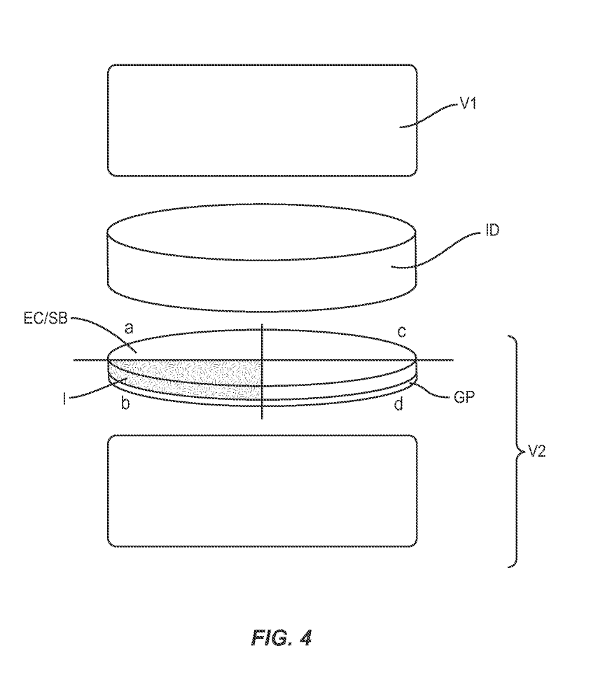

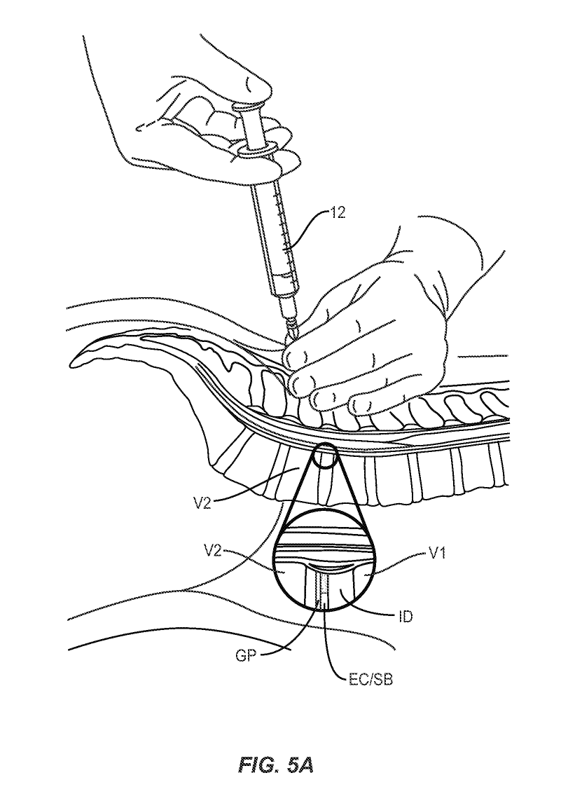

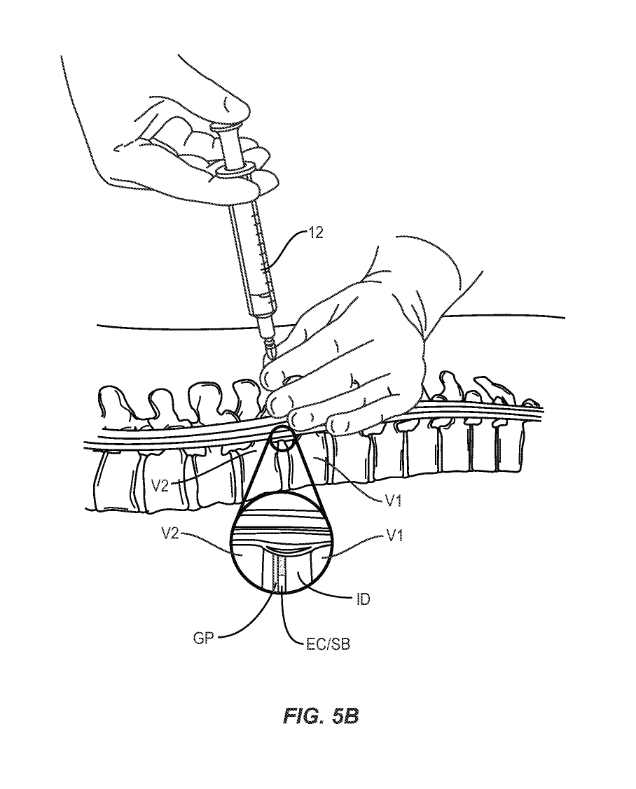

Needle 12, as described herein, is provided with implant I and delivered to the surgical site along a surgical pathway. Needle 12 is delivered to a convex side of vertebra V2 and vertebrae V, and/or separately to a concave side of vertebra V2 and vertebrae V, and/or a first needle 12 is delivered to the convex side and a second needle 12 is delivered to the concave side, as shown in FIG. 3. Needle 12, as described herein and provided with implant I, is delivered to a selected quadrant a, b, c and/or d of vertebra V2 and/or vertebrae V, as shown in FIGS. 4 and 5A. For example, implant I is delivered to the surgical site along a surgical pathway to selected quadrant b, as shown in FIG. 4, adjacent to a first side, such as, for example, a convex side of vertebra V2 and vertebrae V, as shown in FIG. 3. In some embodiments, as shown in FIG. 5A, implant I is delivered adjacent a concave side of vertebrae V in the lumbar region of the spine. In some embodiments, as shown in FIG. 5B, implant I is delivered adjacent a concave side of vertebrae V in the thoracic region of the spine. Implant I is configured for delivery into epiphyseal cartilage EC and subchondral bone SB comprising trabecular bone disposed above growth plate GP adjacent the convex side, as shown in FIG. 4 (epiphyseal cartilage EC, subchondral bone SB and growth plate GP of vertebrae V2 are shown and similar portions of vertebra V1 are not shown for illustration purposes). Tip 20 engages and forms a pathway for needle 12.

Needle 12 is configured for intra-penetration of vertebra V2 and penetrates a portion of vertebra V2, which comprises epiphyseal cartilage EC and subchondral bone SB above growth plate GP and is disposed between a portion of cortical bone of vertebra V2 and an adjacent intervertebral disc ID. Implant I is dispensed through openings 32, 32a, as described herein, such that implant I flows out of openings 32 and/or 32a intra-vertebrally into a selected portion of vertebra V2, which includes epiphyseal cartilage EC and subchondral bone SB. In some embodiments, implant I is dispensed through openings 32, 32a such that implant I flows out of openings 32 and/or 32a in a planar or flat configuration with epiphyseal cartilage EC and subchondral bone SB. In some embodiments, the entirety of implant I is injected intra-vertebrally within vertebra V2. In some embodiments, implant I is not injected and/or disposed outside of vertebra V2 or within intervertebral disc ID.

Implant I is injected within quadrant b of epiphyseal cartilage EC and subchondral bone SB for implantation therewith. In some embodiments, implant I, as described herein, stiffens epiphyseal cartilage EC and subchondral bone SB to apply pressure to growth plate GP adjacent the convex side of vertebra V2 and vertebrae V. In some embodiments, implant I, as described herein, causes the tissue adjacent epiphyseal cartilage EC and subchondral bone SB to swell and apply pressure to growth plate GP adjacent the convex side of vertebra V2 and vertebrae V. As such, implant I applies pressure to growth plate GP to prevent and/or inhibit growth along the convex side C of vertebrae V. In some embodiments, an implant may be injected with epiphyseal cartilage EC and subchondral bone SB to accelerate growth of a vertebral body adjacent a concave side of vertebra V2 and vertebrae V. In one embodiment, implant I is injected into one or more quadrants of vertebra V2 and vertebrae V to create asymmetric growth and correct three dimensional deformities.

As shown in FIG. 3, the components of system 10 are attached with the convex side of vertebrae V to prevent growth of a selected section of vertebrae V, while allowing for growth and adjustments to a second side, such as, for example, a concave side of vertebrae V to provide treatment. Compression of vertebrae V occurs along the convex side due to the application of pressure to growth plate GP according to the injection of implant I, as described herein.

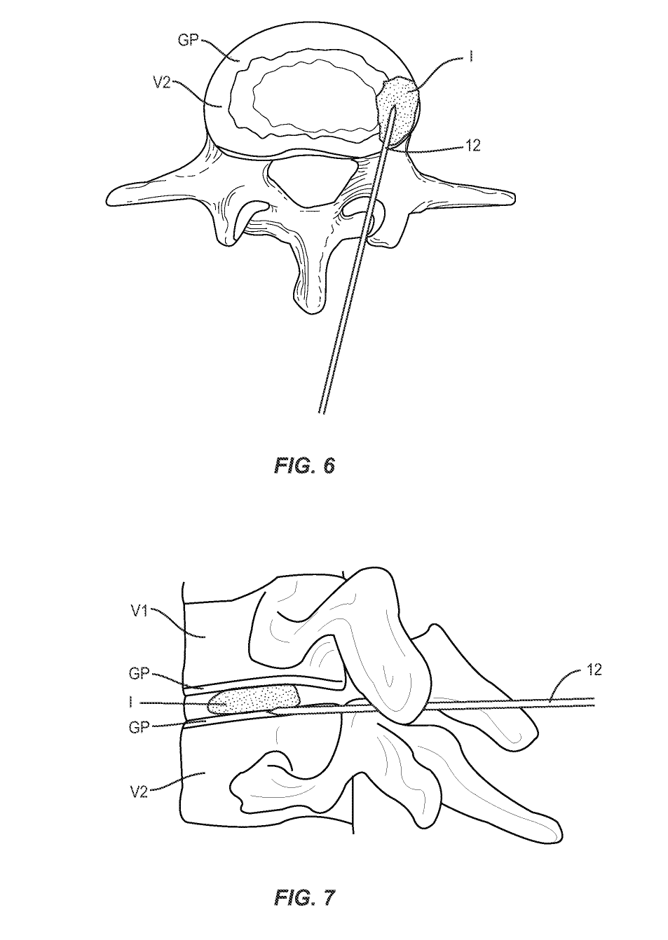

In one embodiment, as shown in FIGS. 6 and 7, a planar cavity is created in the selected portion of vertebra V2 along a posterior approach with needle 12 for disposal of implant I. Other components of system 10 may be delivered to the surgical site, for example, spinal plates, spinal rods and/or screws.

In one embodiment, system 10 includes an agent, which may be disposed, packed, coated or layered within, on or about the components and/or surfaces of system 10. For example, needle 12 can comprise one or a plurality of surface treatments and/or coatings including the agent. In some embodiments, the agent may include bone growth promoting material, such as, for example, bone graft to enhance fixation of the fixation elements with vertebrae V.

In some embodiments, the agent may include therapeutic polynucleotides or polypeptides. In some embodiments, the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of system 10 can be made of radiolucent materials such as polymers. Radiomarkers may be included for identification under x-ray, fluoroscopy, CT or other imaging techniques. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of system 10. Upon completion of the procedure, the surgical instruments, assemblies and non-implanted components of system 10 are removed from the surgical site and the incision is closed.

In some embodiments, the components of system 10 may be employed to treat progressive idiopathic scoliosis with or without sagittal deformity in either infantile or juvenile patients, including but not limited to pre-pubescent children, adolescents from 10-12 years old with continued growth potential, and/or older children whose growth spurt is late or who otherwise retain growth potential. In some embodiments, the components of system 10 and method of use may be used to prevent or minimize curve progression in individuals of various ages.

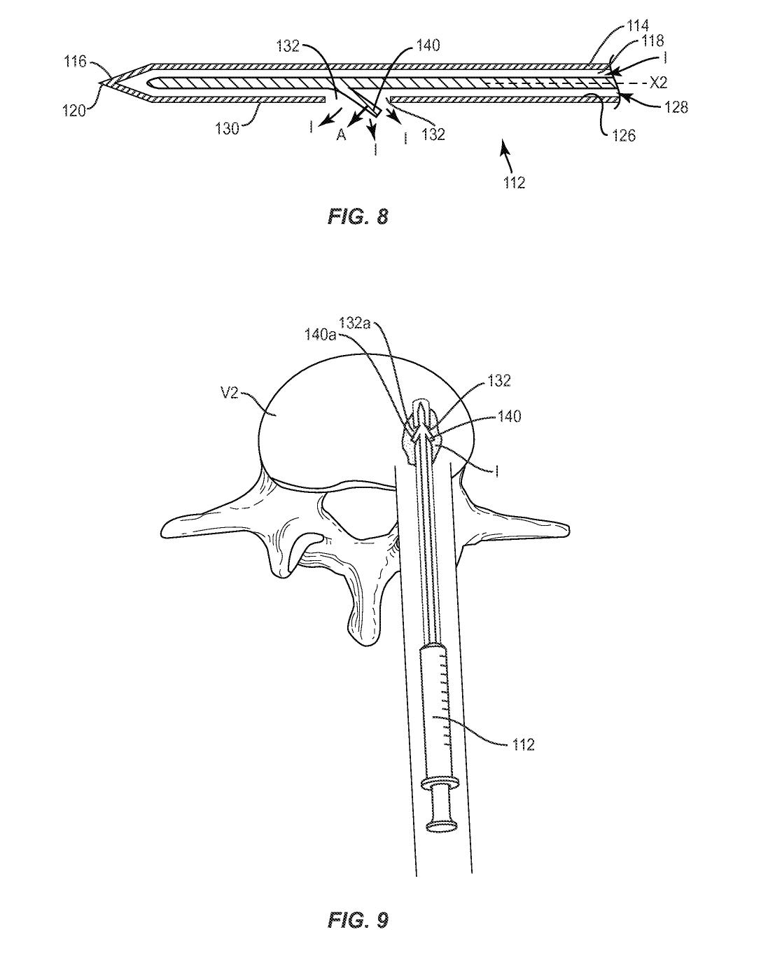

In one embodiment, as shown in FIGS. 8 and 9, system 10, similar to the systems and methods described with regard to FIGS. 1-7, comprises an intra-vertebra needle 112 extending between an end 114 and an end 116. Needle 112 defines a longitudinal axis X2. End 114 includes an opening 118 configured for connection with a dispenser of implant I, similar to that described herein. End 116 includes a pointed tip 120 configured to penetrate and/or engage tissue, as described herein.

Needle 112 includes a surface 126 that defines a passageway 128 configured for disposal of implant I. Needle 112 includes a surface 130 that defines at least one lateral opening 132. In some embodiments, lateral openings 132 may be disposed at alternate orientations, relative to axis X2, such as, for example, transverse, perpendicular and/or other angular orientations such as acute or obtuse, co-axial and/or may be offset or staggered.

Opening 132 includes a flange, such, as for example, a spring biased blade 140. Blade 140 is configured for movable disposal within opening 132. Blade 140 is disposed within passageway 128 and is biased in an orientation, as shown by arrow A in FIG. 8, such that blade 140 extends a distance past surface 130 in opening 132. In some embodiments, blade 140 includes an elastic material, such as, for example, Nitinol and/or PEEK.

In one embodiment, needle 112 includes a plurality of openings 132a, 132 disposed on opposite sides of needle 112. Openings 132 are configured to deliver implant I such that implant I flows through openings 132 and along blades 140, 140a into a convex side of each of a plurality of vertebrae to prevent growth of vertebrae of a selected section of the spine, similar to that described herein. Withdrawal of needle 112 from the surgical site while injecting and/or delivering implant I creates a planar swath of implant 12 in vertebra V2, as shown in FIG. 9.

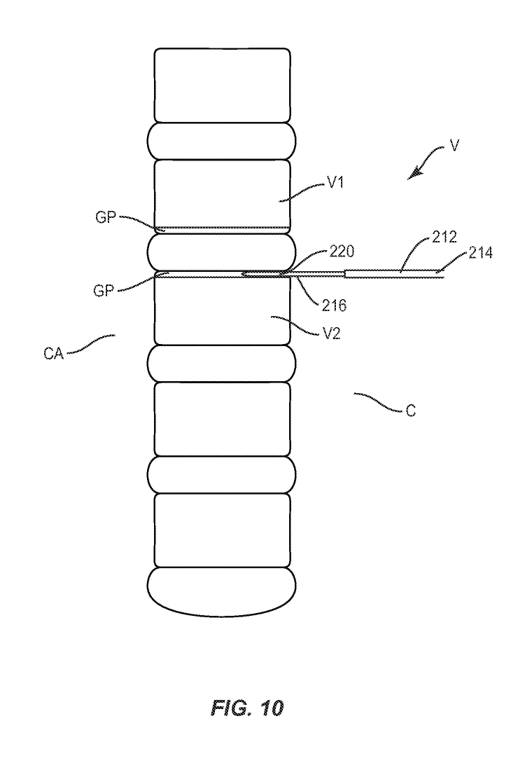

In one embodiment, as shown in FIG. 10, system 10, similar to the systems and methods described with regard to FIGS. 1-7, includes a surgical instrument, such as, for example, a cryogenic probe 212. Probe 212 extends between an end 214 and an end 216. End 214 is configured for connection to a power source (not shown). End 216 includes a cryogenic tip 220 configured to modulate and/or modify tissue growth, similar to that described herein. In some embodiments, tip 220 ablates a selected quadrant of growth plate GP, as described above, of vertebra V2 and/or vertebrae V along convex side C of vertebrae V.

In some embodiments, probe 212 modulates and/or modifies tissue growth of vertebrae V via ablation of vertebral tissue. In some embodiments, probe 212 ablates vertebral tissue by employing cryo-ablation, which removes heat from the vertebral tissue to destroy and/or damage selected vertebral tissue, such as, for example, a selected quadrant of growth plate GP. In some embodiments, end 214 is connected to a pressurized refrigerant source, such as, for example, liquid nitrous oxide. The liquid refrigerant travels through tubing of probe 212 to tip 220. The liquid refrigerant vaporizes as it is sprayed into tip 220 and absorbs heat from surrounding vertebral tissue, thereby cooling and freezing selected vertebral tissue. In some embodiments, tip 220 discharges liquid refrigerant to adjacent the selected vertebral tissue to create temperatures as low as −75° C. adjacent the selected vertebral tissue.

In some embodiments, cryogenic probe 212 is employed with a navigated, as described herein, thora-scopic approach for treatment of vertebrae V. Probe 212 ablates a selected quadrant of growth plate GP on convex side C of vertebra V2 to modulate and/or modify tissue growth such that growth of convex side C of vertebrae V is inhibited and concave side CA continues to grow and straighten vertebrae V. In some embodiments, probe 212 is utilized on alternating growth plates GP along convex side C allowing for growth on concave side CA, while inhibiting growth on convex side C for correction of vertebrae V.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating a spine, the method comprising the steps of:
providing at least one bone growth modification implant; and
disposing the entire bone growth modification implant within a selected portion of a vertebra of a spine.

2. A method as recited in claim 1, wherein the at least one bone growth modification implant includes at least one bone growth inhibiting implant.

3. A method as recited in claim 2, wherein the at least one bone growth inhibiting implant includes an absorbent fluid configured to apply pressure to the vertebra.

4. A method as recited in claim 1, wherein the at least one bone growth modification implant includes at least one bone growth inhibiting implant comprising a bone cement configured to stiffen the vertebra.

5. A method as recited in claim 1, wherein the at least one bone growth modification implant includes at least one bone growth promoting implant.

6. A method as recited in claim 1, wherein the selected portion includes trabecular bone above a growth plate of the vertebra.

7. A method as recited in claim 6, wherein the at least one bone growth modification implant stiffens the trabecular bone to apply pressure to the growth plate.

8. A method as recited in claim 1, wherein the selected portion is disposed along a convexity of the spine.

9. A method as recited in claim 1, wherein the selected portion includes epiphyseal cartilage above a growth plate of the vertebra disposed along a convexity of the spine such that the at least one bone growth modification implant stiffens the epiphyseal cartilage to apply pressure to the growth plate.

10. A method as recited in claim 1, wherein the step of disposing includes injecting the at least one bone growth modification implant into the selected portion.

11. A method as recited in claim 1, wherein the step of disposing includes positioning the at least one bone growth modification implant adjacent one or more quadrants of the vertebra for asymmetric growth of the vertebra.

12. A method as recited in claim 1, further comprising a step of delivering the at least one bone growth modification implant to a surgical site including the selected portion via a posterior percutaneous surgical pathway.

13. A method as recited in claim 1, further comprising a step of delivering the at least one bone growth modification implant to a surgical site including the selected portion via a lateral percutaneous surgical pathway.

14. A method as recited in claim 1, further comprising a step of providing an injection device including an intra-vertebra needle that delivers the at least one bone growth modification implant to the selected portion.

15. A method as recited in claim 1, further comprising a step of creating a planar cavity of the selected portion with the intra-vertebra needle for disposal of the at least one bone growth modification implant.

16. A spinal implant system comprising:
at least one bone growth inhibiting implant; and
an injection device including an intra-vertebra needle having at least one lateral opening configured to deliver the at least one bone growth inhibiting implant within a selected portion of a vertebra of a spine.

17. A spinal implant system as recited in claim 16, wherein the intra-vertebra needle includes a spring biased blade movable in the at least one lateral opening.

18. A spinal implant system as recited in claim 16, wherein the at least one lateral opening includes a plurality of openings disposed in a linear orientation.

19. A spinal implant system as recited in claim 16, wherein the at least one lateral opening includes a first plurality of openings disposed in a linear orientation along a first side of the intra-vertebra needle and a second plurality of openings disposed in a linear orientation along a second side of the intra-vertebra needle.

20. A method for treating a spine, the method comprising the steps of:
providing at least one bone growth inhibiting implant;
delivering the at least one bone growth inhibiting implant to a surgical site including a selected portion of a vertebra of a spine; and
injecting the bone growth inhibiting implant within a selected portion of a vertebra of a spine.
